(19) United States
(12) Patent Application Publication
(10) Pub. No.: US 2025/0020612 A1
(43) Pub. Date: Jan. 16, 2025

(54) METHOD AND SENSOR FOR DETERMINING A PLASMA-RELATED ANALYTE CONCENTRATION IN WHOLE BLOOD

(71) Applicant: SensLab Gesellschaft zur Entwicklung und Herstellung bioelektrochemischer Sensoren mbH, Leipzig (DE)

(72) Inventors: Bernd Gründig, Frohburg (DE); Manfred Rost, Leipzig (DE); Frank Weissenborn, Leipzig (DE); Jörg Weitzenberg, Halle (DE); Sybille Wollermann, Böhlen (DE); Anke Reimer, Grimma (DE); Marion Hänsler, Leipzig (DE); Christopher Pöhlmann, Leipzig (DE)

(21) Appl. No.: 18/898,602

(22) Filed: Sep. 26, 2024

Related U.S. Application Data
(63) Continuation of application No. PCT/EP2023/053223, filed on Feb. 9, 2023.

(30) Foreign Application Priority Data
Mar. 28, 2022 (DE) 102022107214.2

(51) Int. Cl. G01N 27/327; G01N 33/49

(52) U.S. Cl. CPC G01N 27/3272; G01N 33/49

(57) ABSTRACT

The invention describes a method and sensor for determination of a plasma-related analyte concentration in whole blood. Using a temperature-measuring resistor applied to a carrier of the sensor, an ambient temperature is determined with which hematocrit, interference concentration of electrochemically active substances, and an analyte concentration of the whole blood sample are determined in temperature-corrected manner. The temperature-corrected hematocrit is then used in order to determine a plasma-related hematocrit- and temperature-corrected analyte concentration and a plasma-related hematocrit- and temperature-corrected interference concentration. Thereupon, the analyte concentration is determined by subtracting the hematocrit- and temperature-corrected interference concentration from the hematocrit- and temperature-corrected analyte concentration.

### CROSS-REFERENCE TO RELATED APPLICATIONS

This application claims priority to, and is a 35 U.S.C. § 111(a) continuation of, PCT international application number PCT/EP2023/053223 filed on Feb. 9, 2023, incorporated herein by reference in its entirety, which claims priority to German patent application serial number 102022107214.2 filed on Mar. 28, 2022, incorporated herein by reference in its entirety.

### TECHNICAL FIELD

The invention relates to a method and a sensor for determination of a plasma-related analyte concentration in whole blood.

### BACKGROUND

Whole blood measurements of analytes such as lactate or glucose using enzymatic-voltammetric sensors, in particular single-use (disposable) sensors are influenced by a number of parameters resulting both from environmental conditions and the sample matrix itself. Substantial causes of erroneous measurements may originate from a sample's hematocrit, redox-active endogenous metabolites and medicines and, above all, ambient temperature. Temperature has an influence not only on the enzymatic indication reaction, but also on the viscosity of the sample, the dissolution behavior of the reagent layer, the diffusibility of the substances involved in the indication reaction, including the diffusion-impeding effect of the hematocrit, and also the reference potential.

When designing single-use sensors with temperature correction, a calibration curve is usually recorded with spiked whole blood samples at a standard temperature, for example 25° C., on which every measurement is subsequently based. In parallel, a bundle of calibration curves is repeated at temperatures that are to be expected in the subsequent measuring environment. The temperature range is conventionally between 5° C. and 40° C. An ambient temperature lower than the nominal temperature causes a smaller signal current at a lower base current value, while an ambient temperature higher than the nominal temperature causes a larger signal current at a higher base current value. The resultant base current and slope values of the calibration curves, which were then obtained for selected temperature values in this range, are used, together with the standard calibration curve parameters, to set up an algorithm with which the current concentration value can be corrected for the temperature-related deviation. For this purpose, however, it makes sense for the current ambient temperature to be known or for it to be possible to sense it as accurately and as close to the sensor as possible.

The most frequently encountered technical solution for compensating for the influence of temperature involves using a miniaturized Pt100 temperature sensor arranged in the hand-held measuring device in the immediate vicinity of the inserted disposable sensor and the calibrated temperature measurement from which is used to correct the measured value via an algorithm. Since the measuring device, unlike the disposable sensor, has a relatively high heat capacity, considerable differences can arise, particularly in the event of rapid changes in ambient temperature, between the temperature measured on the device and the temperature in the sensor measuring chamber due to heat generated by components or after charging of an internal storage battery. In addition, depending on the time between puncture sampling and application of the sample onto the sensor and in the event of the ambient temperature deviating significantly from the standard measuring temperature, the drop of blood can result in a measurement temperature on the sensor that deviates considerably from the temperature sensed on the measuring device. In both cases, temperature compensation of the measured values will be erroneous.

Further known prior art solutions describe the use of an amperometric two-electrode arrangement comprising a working electrode (anode) and reference electrode (cathode) that is applied separately next to the amperometric measuring system and is not covered with the detection reagent. Once an extremely high positive polarization voltage in the range from 1 to 3 V has been applied, a signal current should be generated, the level of which is independent of hematocrit (Hct) and analyte and substantially only dependent on sample or ambient temperature. Reagent diffusing onto the “temperature electrodes” after filling of the measuring chamber can prove problematic here.

It is further known to establish a defined relationship between the ambient temperature that is measured via a physical characteristic of the sample using additionally arranged electrodes, and optionally the temperature measured by the measuring device in order to compensate for the influence of temperature.

It is likewise known that, by using a temperature sensor in the instrument but in combination with a series of heat-generating pulses defined in terms of duration and amplitude which, via metallic conductor strips on the disposable sensor, result in a defined temperature rise, it is possible to determine the probable temperature at the indication region of the sensor from the difference. In further cases, a temperature sensor is positioned on the underside of the sensor substrate as part of the sensor contact means, the value from which sensor is also sensed during sample measurement and is used to correct the temperature-dependent measured value deviation.

A technical solution for a disposable sensor for voltammetric measurement of analyte concentration uses admittance and phase angle measurements in which up to four different frequencies of up to 20 kHz are briefly applied, with admittance being dependent on hematocrit and temperature while phase angle has been found to be dependent only on hematocrit. By using different frequencies to increase reliability, empirically established dependences of the two values can be used to create an algorithm that establishes the temperature of the reaction zone in addition to the hematocrit, such that the two variables can be used to correct the enzymatic-voltammetric determination of the analyte.

Furthermore, it is possible to establish a temperature-dependent measurement signal after application of the polarization voltage by reducing the latter in the first step below a limit value required to maintain the electrochemical indication reaction and measuring an offset current, this being is followed by a further reduction of the polarization voltage to a still lower level, such that a second offset current is measured. The difference between the two offset currents is temperature-dependent, such that, given appropriate calibration, a temperature value is obtained that is used for compensation of the temperature-dependent indication reaction. However, it cannot be ruled out that the hematocrit of the sample will create an additional dependence.

A further cause of erroneous measurements may originate from the hematocrit of the blood sample. The hematocrit (Hct) is the proportion by volume of red blood cells in the blood, these cells making up approx. 99% of the cellular components in whole blood. In healthy adults, the hematocrit is between 40% and 48% (men) or 36-42% (women). However, values between 20% and 70% can also occur depending a test subject's genetic predisposition, their age, gender, state of health and level of physical activity.

Red blood cells are oval cells with a diameter of 2 to 30 μm which, at 74.6 g/dL (W/V), have a distinctly lower water content than plasma (94.2 g/dL, W/V). For example, when measuring glucose, which can pass unimpeded through the membrane of red blood cells, in whole blood with an Hct in the normal range, this fact can result in a measured value that is approx. 10% lower than a plasma measurement.

If the measurement is of metabolites that enter red blood cells via transporter proteins, such that there is a difference between their concentration in the plasma and in the red blood cells, the deviation can be significantly greater. For example, the concentration of lactate in red blood cells is stated to be between 50% and 70% of the plasma value.

When designing single-use sensors, the measured value for the normal hematocrit range is calibrated to the whole blood value, such that Hct-related measurement errors are within a defined, previously established concentration range and are set out in the specification.

For hematocrit values that are outside the normal range, this measurement error caused by red blood cell volume can increase considerably.

Furthermore, in voltammetric measurements by single-use sensors that have an oxidoreductase for the specific reaction with the target analyte and a redox mediator for electron transfer between the oxidoreductase and the working electrode surface, Hct contributes to further error-causing effects: (i) Due to the lower conductivity of red blood cells compared to plasma, the steady-state measurement in an electrochemical measuring cell leads to an increase in measuring cell resistance and thus to a lower current. This effect can be largely compensated for by using a potentiostatic three-electrode arrangement. (ii) The “particulate nature” of red blood cells impedes diffusion processes which affect both the enzymatic substrate or analyte and the electron mediator that transports the electrons to or away from the enzymatic redox reaction between the enzyme and the electrode surface. A similar situation applies to the corresponding mass transfer at the reference or counter-electrode. As a result of the impeded diffusion, a lower Faraday current is generated compared to the plasma measurement.

Accordingly, the measured value of an analyte concentration of samples with a high hematocrit value will be too low while that from samples with a low hematocrit value will be too high.

It is known to compensate for the hematocrit value by supplying two electrodes of the amperometric measuring chain with an AC voltage component in parallel to the DC voltage so as to enable an impedance measurement. A factor for hematocrit dependence is established from the admittance and/or phase angle, which can if necessary be established at different frequencies, and used to correct the amperometric measurement. It is also known to carry out two impedance measurements in the sample-measuring chamber of a disposable sensor, the frequencies of which can differ by a factor of two to 100 and the second frequency is greater than 20 kHz. The impedance measurement is made on a two-electrode arrangement that is arranged in the vicinity of the sample-receiving opening, such that a first impedance measurement is carried out immediately on sample input and before the reagent mixture has dissolved and a second impedance measurement is carried out after the reagent mixture has dissolved. A multiple regression analysis is carried out on the basis of the two measured values using empirically established functions, which are also dependent on cell parameters and the reagent system, and in this way the hematocrit value, which is used for compensation of the analyte-dependent wanted signal, is calculated.

It is furthermore known in the prior art to provide a measuring chamber with two measuring zones, each with two pairs of electrodes arranged in succession, the first two of which are covered with an analyte-detecting reagent layer and form an amperometric two-electrode arrangement. This measuring zone is followed by a separating element in the form of a polymer layer, which is then followed by the second measuring zone with the second pair of electrodes. The latter are operated with an AC voltage and are used for measurement of the conductivity of the sample. The hematocrit-dependent measured conductivity value is used for compensation of the hematocrit-dependent amperometric concentration measurement.

A sensor arrangement with a capillary gap measuring chamber is furthermore known in which two lateral spacer walls are made of conductive material to which an alternating current measurement is applied in order to determine the conductivity of the sample and thus the hematocrit value, which is used to correct the analyte-dependent amperometric measurement. It is also known to provide a capillary measuring chamber with three successively arranged identical circular electrodes, which constitute two working electrodes and the reference electrode and are microfluidically connected together via a gap-like channel arranged thereover. One of the working electrodes and the reference electrode have an identical reagent mixture, such that, once the sample has been drawn up via the connecting channel between the two electrodes, the hematocrit-dependent conductivity of the sample is measured and is used to correct the amperometric measurement.

However, when measuring the conductivity of the sample, it must also be borne in mind that analytes such as lactic acid or the resulting lactate in dissociated form are present and cause an increase in conductivity with increasing concentration, such that the Hct, which is determined via a conductivity measurement, may be erroneous if no account is taken of analyte concentration.

Other known solutions make use of pulse potential and potential scanning methods, special electrode arrangements in a measuring cell and additional redox indicators to quantify diffusion-dependent influences in voltammetric measurements in blood samples, such as those originating from the hematocrit. For example, diffusion-determining scan phases can be used during a cyclic scan to establish the hematocrit fraction, for example by determining the ratio of peak current to plateau current, which is analyte-independent and essentially depends only on the diffusion-determining component of the sample.

Furthermore, when using differential pulse voltammetry to determine concentration in blood, the resulting current curves can be evaluated while the current is still below the diffusion-limiting peak current, such that the diffusion influence of hematocrit on the measurement result can be largely excluded. In particular, short high-frequency pulses are applied to maintain the diffusion layer of the reagent layer.

Square wave voltammetry (SWV), in which an additional redox mediator is used to detect a redox-mediated enzymatic detection reaction in blood samples, is also known. The formal redox potential of this mediator is sufficiently far away from that of the indication mediator that it cannot enter into a reaction with either the indication mediator or the oxidoreductase. This redox mediator acts as an internal standard which, like the signal-generating system, is influenced by hematocrit and temperature via the diffusion rate, but is detected independently of the substrate and can therefore be used to obtain hematocrit information.

The mobility or diffusibility of the redox mediator, which is substantially influenced by Hct, can be established as follows: after voltammetric-enzymatic measurement of analyte concentration, the polarization voltage is switched off and the voltage drop, which is substantially determined by the mobility of the redox mediator, is tracked potentiometrically between the working and reference electrodes. The rate at which the voltage drops is thus a variable for indirectly determining the hematocrit value, which is used to correct the concentration measurement and may optionally also be used to differentiate between the control solution and the sample. The mobility of the redox mediator can be determined by applying it to the reference electrode and measuring the resulting reduction current after application to the working electrode of a negative voltage relative to the reference electrode. The measuring current is solely dependent on how quickly the redox mediator can diffuse to the working electrode. The lower the current, the greater the diffusion barrier or the higher the red blood cell content in the sample, such that a hematocrit-dependent signal is obtained that can be used to correct the actual concentration-dependent measurement signal.

The intention is to use the hematocrit-related change in viscosity of the sample, which can lead to correspondingly different filling times of the measuring chamber of a capillary gap sensor, to establish a hematocrit-dependent signal, which is error-prone in particular in the case of fluctuating ambient temperatures.

In addition to using the electron mediator required for the enzymatic redox reaction, other solutions describe using a second redox mediator that does not react with the first and differs therefrom by at least 200 mV with regard to its formal redox potential. Amperometric detection of the reduction or oxidation of this mediator will then substantially depend on its diffusibility in the measurement cell, which is determined by the hematocrit concentration. The resultant hematocrit-dependent current signal, which is quantified using chronoamperometry, SWV, DPV (Differential Pulse Voltammetry) or CV (Cyclic Voltammetry) and is Hct-calibrated according to an empirically established algorithm, is additionally used for compensation of the hematocrit dependence of the analyte-dependent signal. Another technical solution is based on the charging current immediately after application of the polarization voltage being substantially determined by the hematocrit. If this value is set in relation to the subsequent Faraday current by forming a quotient, a hematocrit-dependent variable is also obtained.

Other amperometric measurement procedures known from technical solutions evaluate transitional states after application of the polarization voltage, which is varied in amplitude and sign, in order to establish the Hct fraction in addition to the analyte-dependent signal.

In a further method, two polarization voltages of opposite sign are applied in succession over a period of 1 to 20 s, the first voltage being negative and the second positive. Using a three-dimensional calibration graph, a hematocrit-corrected analyte concentration is established from the resultant “transient currents”, which are dependent on analyte concentration and the electrochemical cell constant and thus on the Hct. Specifically, the graph is based on the ratio of negative and positive current values and analyte concentration curves that are calibrated against a reference system, each of which was recorded at different hematocrit values. The hematocrit-dependent information is obtained from the fact that, immediately after application of a suitable polarization voltage, the resulting current pulse is initially substantially composed of charge current and a Faraday current component and toward the end of the pulse the Faraday component, which is characterized by the Cottrell equation, predominates. Since the effective diffusion coefficient of the analyte or of a mediator involved in the indication reaction is influenced by the hematocrit, it is possible by suitably calculating a ratio of current portions of the initial signal at a specific polarization voltage or of two successively applied polarization voltages with opposite signs to establish a hematocrit-dependent value that can be used to correct the wanted signal. However, since the influence of hematocrit varies depending on the level of the anodically generated analyte current, the analyte concentration must additionally be taken into account when calculating the hematocrit correction. This fact, which must be taken into account for every electrochemical cell constant, explains the need for a three-dimensional curve bundle.

In order to obtain an Hct-independent analyte measurement using an amperometric two-electrode arrangement consisting of a microelectrode array as the working electrode and a reference electrode, the polarization voltage is applied up to three times at intervals each of a duration of between 0.1 s and 1 s with “recovery phases” therebetween. The polarization voltage is applied close to 0 mV over a duration of 0.05 s and 1 s in each case. The resultant chronoamperometric current curve portion is recorded, wherein the logarithmic current values at the end of the respective interval are used for evaluation by plotting them against the logarithm of the measuring times. It has been found that the resultant point of intersection with the y-axis corresponds to a virtually hematocrit-independent wanted signal.

Another procedure using this amperometric electrode arrangement is directed toward measuring a “test current value” immediately after switching on the polarization voltage and once a steady state current profile has been reached. Plotting the respective quotients against the inverse square root of time gives rise to a linear slope that corresponds to the diffusion coefficient and is used for hematocrit-compensated calculation of the analyte value.

A more general procedure, which does however also make use of the above-described effects, is based on an amperometric two-electrode arrangement of an anode and cathode that are arranged plane-parallel to one another, the resultant gap constituting the measuring chamber. Polarization voltages of opposite sign are applied in two successive intervals. The level of the voltage, referred to as the test potential, which is between −100 mV and −600 mV or +100 mV and +600 mV, is intended to just enable partial reduction or oxidation on the electrode surfaces. During the first interval, a voltage is applied that brings about partial oxidation of the reduced mediator on the second electrode, i.e., the first electrode is cathodically active and reduces the mediator. During the second interval, the mediator, which has optionally been reduced by the enzymatic indication reaction, is partially oxidized on the first electrode. Accordingly, relative to the first electrode, it is for example firstly a negative and then a positive potential that is applied. A provisional measured value for analyte concentration is first established from the signal current resulting from one of the two intervals. The hematocrit-dependent source of error is then established and the analyte value is corrected, taking account of previously established factors such as different concentration ranges for analyte and hematocrit values and evaluative criteria for high or low analyte concentration at high or low hematocrit values, which are based on different, empirically established functions.

In other solutions, two successive polarization voltages with the same polarity are likewise applied to an amperometric two-electrode arrangement, wherein a low anodic voltage between 10 mV and 150 mV is applied within the first second after triggering followed by a higher anodic voltage between 250 mV and 350 mV. The resultant current pulse 450 ms to 530 ms after triggering is evaluated to determine the hematocrit and the subsequent pulse with the higher polarization voltage is used to determine a provisional analyte concentration value.

The hematocrit is determined using a curve bundle previously established with defined analyte and hematocrit concentrations. Different, empirically established equations, which are used to correct the hematocrit of the measured analyte concentration, were established for each of three prestored concentration portions covering the entire analytically relevant range for the analyte.

However, the drawback of this latter method is that it is based on complex calculations using empirically established three-dimensional curve bundles that have to take account of the diffusion-dependent Faraday current component of the signal current or the level of analyte concentration.

A number of technical solutions are known that are intended to minimize, compensate for or prevent the influence of electrochemically active substances from metabolic reactions or medicines. Practical solutions describe additional protective layers, semipermeable or porous membrane layers that are applied over the reaction layer and are intended to prevent or at least greatly reduce diffusion of interfering substances to the electrode system during the comparatively short measuring time. However, it is more effective to detect the interfering substances separately, in particular for patients in a clinical setting where little-known electrochemically active substances from administered medicines or combined effects for which the diffusion barrier layers are not designed are to be expected.

A number of disclosures make use of an additional working electrode that is operated at an overvoltage, such that the interfering substances are more strongly oxidized on the electrode than is the reduced mediator. A factor established according to an empirical equation from the measurement signals of the two working electrodes is used to correct the measured lactate value. However, this approach fails to take account of the fact that electrochemically active substances are not only oxidized directly on the surface of the working electrode, but also react with the redox mediator.

One of the first commercial disposable sensors based on an electrochemical enzymatic indication reaction uses a multilayer fabric structure with hydrophilic and hydrophobic fabric properties over an electrode arrangement. A sample-receiving opening is provided in the upper surface layer. The first fabric layer is additionally impregnated with red blood cell-aggregating polymer components to ensure effective Hct retention. An additional working electrode that is coated with an enzyme-free reagent is used for measurement of electrochemically active, interfering components. This variant still required a comparatively large sample volume and a long measurement time of 20 s due to the time-dependent aggregation.

One known technical solution uses disposable sensors for determining glucose, lactate and creatinine in a capillary gap arrangement and an additional working electrode that is exclusively used for sensing interference currents from the oxidation of ascorbate, uric acid, bilirubin, acetaminophen or other electrochemically active substances in the blood sample, such that the combined signal current can be corrected by this amount.

It is furthermore known to use an overvoltage in the form of an exciting waveform on the working electrode, such that nonlinear current signals are generated. Resolving the signal distribution in accordance with a vector projection method provides numerous real and imaginary components, a plurality of Fourier coefficients or a plurality of frequencies that are calibrated to a reference current signal of an analyte or interfering components, such that either the analyte itself or the interfering substances are selectively determined.

It is also known to measure transition currents immediately after a change in polarization voltage that is carried out straight after filling of the measuring chambers in order to sense electrochemically active substances. Up to three different polarization voltages of opposite polarity are applied to the working electrode in quick but time-defined succession. Once the respective transient currents have been set, they are related to one another and the proportion of interference currents caused by electrochemically active substances is subtracted.

A drawback of all previously known technical solutions is the fact that indirect measurement methods that are often associated with major scatter of measured values are used to correct interfering influences on the analyte signal. Known technical solutions do not describe the correction of the considerable influence of temperature on determining hematocrit and the concentration of ionically conductive components in the sample, for example an analyte present in dissociated form or endogenous or exogenous metabolites present in dissociated form, on the determination of hematocrit. In addition, only one external temperature sensor is used for temperature measurement that is integrated in a connecting measuring device and can therefore lead to erroneous temperature corrections.

It is therefore an object the invention to overcome the stated disadvantages and to provide a sensor and a method for highly accurate determination of a plasma-related analyte concentration in whole blood in order to enable more accurate enzymatic-voltammetric analyte determination during the measurement procedure while compensating for the actual temperature, the hematocrit of the sample and the electrochemically active substances contained therein, such that these interfering influences on the plasma-related measured analyte value to be determined can be adequately corrected. The following description discloses further objects that are achieved.

The object of the invention is achieved according to the independent claims. The subclaims recite preferred variant embodiments.

### SUMMARY

A first aspect of the invention relates to a method for determining an analyte concentration in whole blood by a sensor. In one step, at least two measuring chambers are filled with a whole blood sample via a sample-receiving zone of the sensor, wherein the at least two measuring chambers are formed on a carrier of the sensor and at least two measuring chambers comprise a first voltammetric three-electrode arrangement, a four-electrode conductivity arrangement, and a second voltammetric three-electrode arrangement. In one step, after filling of the measuring chambers, an ambient temperature is measured by a temperature-measuring resistor applied to the carrier. The method further comprises the step of determination of an ionic conductivity of the whole blood sample with the four-electrode conductivity arrangement. The method further comprises the step of voltammetric determination of an interference charge of electrochemically active substances in the whole blood sample by the first voltammetric three-electrode arrangement. The method further comprises the step of enzymatic-voltammetric determination of an analyte charge of the whole blood sample by the second voltammetric three-electrode arrangement. The method furthermore comprises the determination of a temperature-corrected analyte concentration using prestored calibration curves, the determined ambient temperature, and the determined analyte charge. The method further comprises the determination of a temperature-corrected interference concentration using prestored calibration curves, the determined ambient temperature, and the determined interference charge; and further the determination of a temperature-corrected hematocrit value using prestored calibration curves, the determined ambient temperature, and the determined ionic conductivity. The method further comprises correction of the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value. The method further comprises the step of determination of analyte concentration by subtracting the hematocrit- and temperature-corrected interference concentration from the hematocrit- and temperature corrected analyte concentration.

The analyte may for example be glucose, lactate, or creatinine, wherein the invention is not limited thereto. Electrochemically active substances may for example be ascorbate, uric acid, bilirubin, or acetaminophen. Plasma-related means that the analyte concentration is related to the value of a corresponding plasma sample (hematocrit concentration=0%). Potentiostatic three-electrode arrangements are preferably used as the indication systems for the two voltammetric measurements. On subtraction, a sensitivity factor, set to 1 in exemplary embodiments, that weights the hematocrit- and temperature-corrected interference concentration can preferably be determined. The nominal temperature is typically 25° C. The analyte charge is determined enzymatically while the interference charge is determined nonenzymatically. The simple voltammetric measurement is nonspecific. The enzymatic-voltammetric measurement provides the selective or analyte-specific measurement signal which, due to the voltammetric detection principle, does however have a nonspecific component and is corrected by the simple voltammetric measurement.

For temperature correction, the calibration curves may comprise corresponding prestored, i.e., previously recorded, calibration curves between analyte concentration and temperature, between interference concentration and temperature, and between hematocrit/ionic conductivity and temperature. For hematocrit correction, i.e., plasma-related correction, hematocrit is used in each case using previously established calibration curves/correlation curve bundles between analyte concentration and hematocrit and between interference concentration and hematocrit. The prestored calibration curves can be saved in a memory/data memory to which a process carrying out the method has access.

The first measuring chamber and the second measuring chamber may each preferably have a volume of 0.15 μL to 0.3 μL. The time sequence may be as follows: (i) Temperature measurement can take place on the resistor immediately after filling of the measuring chambers and once time-defined current thresholds of the voltammetric measuring channels have been exceeded over 50 to 1000 ms. (ii) An ionic conductivity measurement for hematocrit determination can follow 0.5 s to 1 s later. (iii) Voltammetric measurement for determining the interference concentration in the whole blood sample can take place between the second and ninth seconds. (iv) An enzymatic-voltammetric measurement of analyte concentration can take place between the third and tenth seconds. The entire measuring process is therefore completed in approximately 10 s, or at least less than 20 s.

The described method is in particular suitable for single-use sensors that are intended to provide clinically relevant and reliable plasma-related concentration values. A further technical advantage is that, thanks to the use of a temperature-measuring resistor applied to, i.e., integrated on, the carrier, the current temperature can be measured highly accurately and with no susceptibility to interference in comparison with the prior art (see above explanations). This accurately measured ambient temperature is then also used to eliminate the temperature-dependent interference from hematocrit and electrochemically active substances in addition to correction of the temperature-dependent enzymatic-voltammetric analyte measurement. The influence of hematocrit and interfering electrochemically active substances is thus corrected more systematically and adequately than in known technical solutions. Because the analyte concentration determination is plasma-related, the disposable sensor is particularly suitable for emergency applications or for rapidly required analyte determinations. In contrast to a clinical setting, in which the hematocrit can be removed from the whole blood before the automated analyte measurements or blood gas analyzers are used, this both requiring a significantly larger sample volume and being more demanding in terms of equipment and time, these stated applications use a whole blood sample with a very small volume for processing the hematocrit and the analyte value is sensed in seconds reliably and in a clinically relevant manner even under field conditions.

The method may preferably further comprise correction of the temperature-corrected hematocrit value to an analyte- and temperature-corrected hematocrit value using prestored calibration curves and the determined temperature-corrected analyte concentration, and/or using prestored calibration curves and the determined temperature-corrected interference concentration. The measured ionic conductivity value can thus be corrected on the basis of ionically conductive components in the sample, in particular for correction of an analyte present in dissociated form or interference concentrations present in dissociated form, such as for example endogenous or exogenous metabolites. The hematocrit value is thus determined more accurately, such that the dependent analyte concentration that is to be determined from this determined hematocrit value and the interference concentration to be subtracted can also be determined more accurately. The calibration curves should be determined between ionic conductivity/hematocrit and analyte concentration, or ionic conductivity/hematocrit and interference concentration. The temperature- and analyte-corrected measured conductivity value can be determined using a previously established, nonlinear calibration curve between hematocrit and ionic conductivity of the sample for establishing the hematocrit value.

The temperature-measuring resistor is preferably a serpentine conductor structure applied to the carrier of the sensor. The serpentine conductive structure allows a sufficient resistance length to be produced on a small carrier area. As a result, measurable temperature-related changes in resistance can be sensed, with only a small proportion of the area of the carrier of the sensor being required for this purpose. In addition, because it only requires a small area, the serpentine conductor structure can be positioned close to the measuring chambers, such that the temperature can be determined accurately and with little susceptibility to interference. This in turn improves the accuracy of temperature compensation, such that the plasma-related analyte concentration to be determined is also determined with greater accuracy. The serpentine conductor structure preferably has a resistance between 100Ω and 2000Ω and a temperature coefficient between 0.4Ω/° C. and 0.7Ω/° C. for temperature-dependent resistance measurement.

The serpentine conductor structure is preferably positioned adjacent to the measuring chambers. As a result, the temperature at the measuring chambers can be measured particularly well. This in turn improves the accuracy of temperature compensation, such that the plasma-related analyte concentration to be determined can also be determined with greater accuracy.

The serpentine conductor structure is preferably positioned in a carrier portion that has a length in relation to the sample-receiving zone that is less than one third, preferably less than one quarter, and still more preferably less than one fifth of the total length of the carrier. This firstly ensures that the temperature is measured close to the measuring chambers. Furthermore, sources of heat interference due to the connection of a measuring device may have a less disruptive influence on the temperature measurement. As a result, the temperature at the measuring chambers can be measured more accurately. This in turn improves the accuracy of temperature compensation, such that the plasma-related analyte concentration to be determined can also be determined with greater accuracy.

Determining the ambient temperature preferably comprises the determination of a first temperature after or during filling of the measuring chambers; determination of a second temperature after determination of the hematocrit value, of the analyte charge, and/or of the interference charge of electrochemically active substances in the whole blood sample, and determination of the ambient temperature by arithmetic averaging of the measured temperatures. As a result, an erroneous temperature determination due to temperature drift during the measuring process can be minimized or reduced by averaging. This in turn improves the accuracy of temperature compensation, such that the plasma-related analyte concentration to be determined can also be determined with a higher accuracy because the undesired temperature drift is compensated or averaged out. The two temperature-dependent resistance measurements on the serpentine conductor structure can take place over a period of 50 to 1000 ms.

The first measuring chamber preferably comprises the first voltammetric three-electrode arrangement and the four-electrode conductivity arrangement; the second measuring chamber preferably comprises the second voltammetric three-electrode arrangement. The method further comprises switching of the electrodes in the first measuring chamber between the first three-electrode arrangement for voltammetric measurement and the four-electrode conductivity arrangement for measurement of ionic conductivity using an integrated or reversibly connected analog switch array. It is consequently possible for one measuring chamber to be designed for two measured variables, namely ionic conductivity and interference concentration. The electrodes present can thus be put to dual/double use. This not only saves electrode material, but also enables a more compact measuring zone, the temperature of which can be more accurately sensed by the serpentine conductor structure. This in turn improves the accuracy of temperature compensation, such that the plasma-related analyte concentration to be determined can also be determined with greater accuracy

The first three-electrode and four-electrode conductivity arrangements preferably comprise a reagent coating comprising a redox mediator, and wherein the second three-electrode arrangement has a reagent coating comprising an oxidoreductase or further catalytically active proteins, and a redox mediator.

A total protein content is preferably determined by the quantity of an oxidoreductase or by an oxidoreductase and one or more additional catalytically active proteins. The oxidoreductase preferably comprises oxidases, peroxidases and/or cofactor-dependent dehydrogenases. The catalytically active proteins preferably comprise hydrolases, proteases, and esterases. The redox mediator preferably comprises a redox-active metal complex, a quinoid redox dye, or an organometallic compound.

The temperature-dependent resistance measurement is preferably made using a two-electrode conductivity arrangement and still more preferably a four-electrode conductivity arrangement. A current feed between 100 μA and 750 μA is preferably provided during the ionic conductivity measurement.

An AC voltage without a DC voltage component is preferably applied for the four-electrode conductivity arrangement, wherein the AC voltage is rectangular, triangular, or sinusoidal with a preferred frequency between 100 Hz and 5000 Hz. Preferably, each electrode can be switched off in a defined manner or be interconnected with other electrodes via an analog switch array.

The method preferably comprises measurement of the ionic conductivity of the whole blood sample by the four-electrode conductivity arrangement, voltammetric determination of the interference charge of electrochemically active substances in the whole blood sample by the first voltammetric three-electrode arrangement, and enzymatic-voltammetric determination of the analyte charge of the whole blood sample by the second voltammetric three-electrode arrangement in steps within a measuring interval of 8 s to 20 s and preferably between 8 and 11 s. The short duration of the measuring process can also reduce temperature interference effects. After filling of the measuring chambers with sample, the timing can be broken down as follows: (i) Measurement of serpentine resistance to determine temperature can take place over 50 ms to 1000 ms. (ii) An ionic conductivity measurement for hematocrit determination can follow 0.5 s to 1 s later. (iii) Voltammetric measurement for determining interference concentration in the whole blood sample can take place in the first measuring chamber between the second and ninth seconds. (iv) An enzymatic-voltammetric measurement of analyte concentration can take place between the third and tenth seconds. (i) A second measurement of serpentine resistance to determine temperature can take place over 50 ms to 1000 ms.

A further aspect of the invention describes a sensor for determination of a plasma-related analyte concentration in whole blood. Said sensor comprises at least two measuring chambers that are fillable with a whole blood sample via a sample-receiving zone of the sensor, wherein at least two measuring chambers are formed on a carrier and comprise a first voltammetric three-electrode arrangement, a four-electrode conductivity arrangement, and a second voltammetric three-electrode arrangement. The sensor further comprises a temperature-measuring resistor applied to the carrier for determining an ambient temperature. The sensor further comprises at least one processor unit that is reversibly connected to the sensor via an electrical contacting means or is integrated on the carrier, and is set up to do the following: control the temperature-measuring resistor in order to measure the ambient temperature after filling of the measuring chambers. Functionality further comprises the step of control of the four-electrode conductivity arrangement in order to determine an ionic conductivity of the whole blood sample. Further included is the step of control of the first three-electrode arrangement in order to voltammetrically determine an interference charge of electrochemically active substances in the whole blood sample. Additionally included is the step of control of the second three-electrode arrangement in order to enzymatic-voltammetrically determine an analyte charge of the whole blood sample. Additionally included is the step of determination of a temperature-corrected analyte concentration using prestored calibration curves, the determined ambient temperature, and the determined analyte charge. Further included is the determination of a temperature-corrected interference concentration using prestored calibration curves, the determined ambient temperature, and the determined interference charge. Additionally included is the determination of a temperature-corrected hematocrit value using prestored calibration curves, the determined ambient temperature, and the determined ionic conductivity. A further step comprises correction of the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value. In addition, the analyte concentration is determined by subtracting the hematocrit- and temperature-corrected interference concentration from the hematocrit- and temperature-corrected analyte concentration. In other words, the processor unit is a processor or microcontroller. The prestored calibration curves can be saved in a memory/data memory to which the processor unit is operatively connected.

The same advantages are achieved as described in relation to the above method, to which reference is here made.

The temperature-measuring resistor is preferably a serpentine conductor structure applied to the carrier of the sensor. The serpentine conductor structure is preferably positioned adjacent to the measuring chambers.

The serpentine conductor structure is positioned in a carrier portion that has a length in relation to the sample-receiving zone that is less than one third, preferably less than one quarter, and more preferably less than one fifth of the total length of the carrier.

The first measuring chamber preferably comprises the first voltammetric three-electrode arrangement and the four-electrode conductivity arrangement, and wherein switching the electrodes in the first measuring chamber between the first three-electrode arrangement for voltammetric measurement and the four-electrode conductivity arrangement for measurement of ionic conductivity is to be carried out using an integrated or reversibly connected analog switch array.

Further preferred embodiments can also be inferred from the above method.

### BRIEF DESCRIPTION OF THE DRAWINGS

The invention is explained in greater detail below with reference to an exemplary embodiment and associated drawings. In the figures:

FIG. 1 shows an inventive sensor according to a preferred embodiment of the invention;

FIG. 2 is a schematic representation of a sensor according to a preferred embodiment of the invention;

FIG. 3 is a schematic representation of a flow chart according to the invention;

FIG. 4 shows an exemplary prestored calibration curve of the measured serpentine resistance value as a function of ambient temperature;

FIG. 5 shows exemplary prestored calibration curves for resistance measurement (1/ionic conductivity) as a function of sample hematocrit at ambient temperatures of 15° C., 25° C. and 45° C. in the first measuring chamber;

FIG. 6 shows exemplary prestored calibration curves for charge values recorded as a function of lactate concentration in the second measuring chamber at temperatures between 5° C. and 45° C.;

FIG. 7 shows an exemplary prestored curve bundle generated from the calibration curves of FIG. 6 by plotting lactate concentration against temperature for various charge values measured in the second measuring chamber;

FIG. 8 shows exemplary prestored calibration curves for charge values as a function of lactate concentration at hematocrit concentrations of 0% (plasma), 19%, 45% and 70% measured in the second measuring chamber;

FIG. 9 shows an exemplary prestored curve bundle generated from the calibration curves in FIG. 8 by plotting lactate concentration against the hematocrit value at various charge values measured in the second measuring chamber, and

FIG. 10 shows exemplary prestored calibration curves for measured charge values against a dilution series of a model solution of 0.9 mM ascorbic acid, 1.03 mM acetaminophen and 1.4 mM uric acid measured in the first and second measuring chambers.

### DETAILED DESCRIPTION

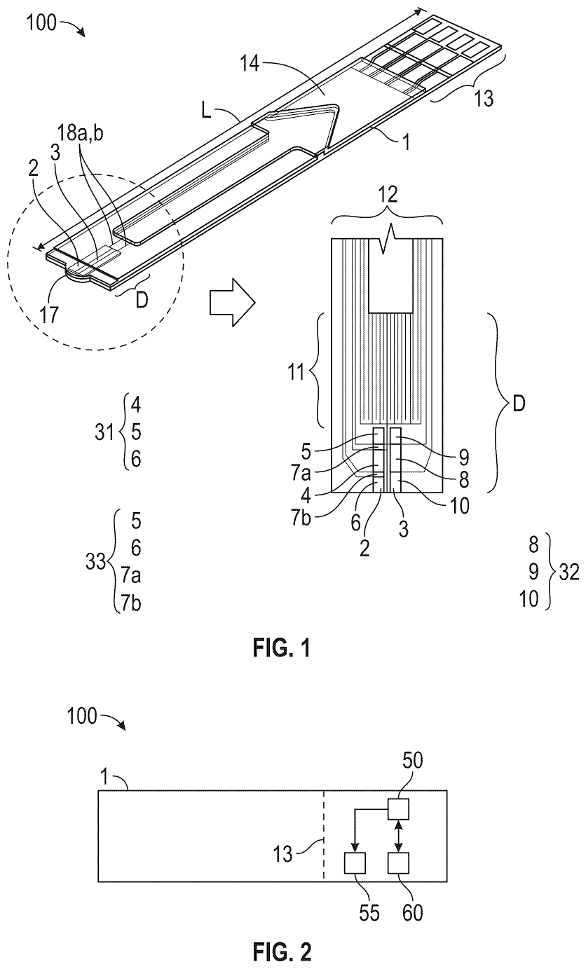

FIG. 1 shows an inventive sensor 100 according to a preferred embodiment of the invention. The sensor 100 is in particular a single-use or disposable sensor.

The sensor 100 comprises a planar carrier 1 extending along a longitudinal axis. Appropriate sensor components are applied to the carrier 1. The carrier material is preferably a plastics material, for example a PET (polyester). The carrier 1 may for example have a thickness/gage of 0.25 mm, wherein the invention is not limited thereto.

The sensor 100 further comprises a sample-receiving zone 17 at a sample-receiving end of the carrier 1, via which a whole blood sample, for example a capillary blood sample, can be guided into measuring chambers 2, 3. Measuring chambers 2, 3 have to this end a common sample-receiving zone 17 and extend parallel to one another. Measuring chambers 2, 3 are further applied to the carrier 1 in liquid-tight manner relative to one another. An electrically insulating coating film 14 can be printed thereunder, said coating film 14 containing two measuring windows parallel to one another that are left blank and thus define or bound the areas of associated electrode arrangements. A serpentine conductor structure of a temperature-measuring resistor 11 can also be left blank. Measuring chambers 2, 3 may preferably be designed to have a capacity between 150 nL and 300 nL. Vent channels 18a,b may also be provided.

In the magnified portion of FIG. 1, electrodes 4, 5, 6, 7a, 7b, 8, 9, 10 are also visible on the carrier 1. A temperature-measuring resistor 11 for measuring ambient temperature is also applied to the carrier 1 of the sensor 100. The positioning on the carrier 1 makes the temperature measurement less susceptible to interference and the distance from the measuring chambers can be kept small. The temperature-measuring resistor 11 is embodied as a serpentine/sinuous conductor structure. As a result, measurable temperature-related changes in resistance can be sensed with sufficient accuracy, with only a small proportion of the area of the carrier 1 of the sensor being required for this purpose.

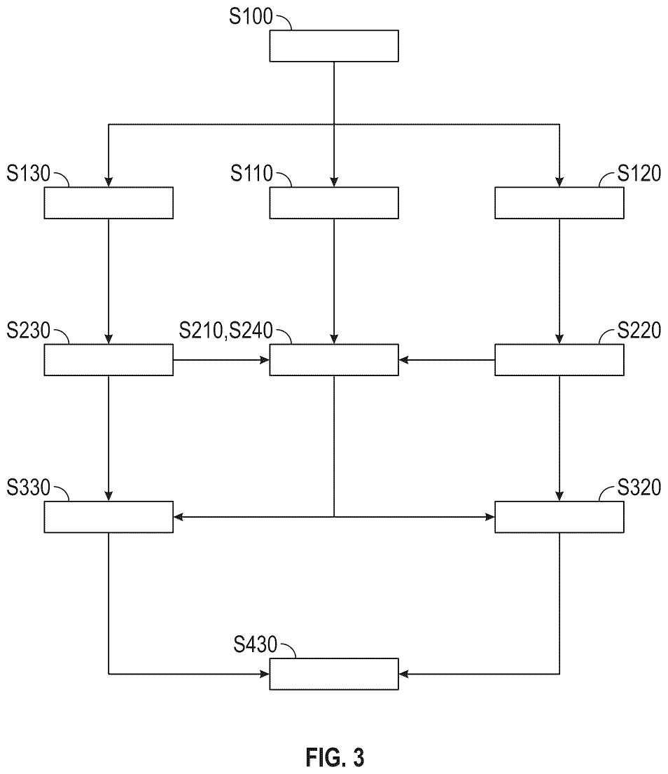

The serpentine conductor structure 11 is positioned (immediately) adjacent to measuring chambers 2, 3 and thus in the immediate vicinity of measuring chambers 2, 3. As a result, the temperature of the measuring chambers 2, 3 can be measured particularly well and reliably. This in turn improves the accuracy of temperature compensation of the required measured variables, which are described in greater detail in particular in relation to FIG. 3.

The serpentine conductor structure 11 is positioned in a carrier portion D of the carrier 1 that has a length that is less than one third, and preferably less than one fifth, of the total length L of the carrier 1. In this way, sources of thermal interference, for example due to the connection and operation of a measuring device, may have a lesser interference effect on the temperature measurement, such that the temperature at measuring chambers 2, 3 can be measured more accurately.

The serpentine conductor structure 11 may be completely covered by the insulating coating 14 or preferably also left blank. Feed lines 12 to electrodes 4, 5, 6, 7a, 7b, 8, 9, 10 and to the serpentine conductor structure 11, which ensure operation of electrodes 4, 5, 6, 7a, 7b, 8, 9, 10 and the serpentine conductor structure 11, are also provided. Electrical contact means 13, in particular contact surfaces, are also applied to the carrier 1 at the opposite end from sample-receiving zone 17. A measuring device, or in particular a processor 50 of a measuring device, can be reversibly connected to the sensor 100 via the electrical contact means 13, as shown schematically in FIG. 2.

In one particular embodiment, the structures can be produced in the following manner. After a sputtering process in which a thin film of an inert metal, for example a layer of gold with a film thickness of 50 nm is applied, a first three-electrode arrangement 31 with working electrode AE1 4, counter-electrode GE1 5 and reference electrode RE1 6 including two additional voltage-tapping measuring electrodes 7a,b and, in parallel, a second three-electrode arrangement 32 with working electrode AE2 8, counter-electrode GE2 9 and reference electrode RE2 10 are ablated using a laser. The serpentine conductor structure 11 including feed lines 12 and electrical contact means 13 can be patterned by ablation in the immediate vicinity.

The electrical contact means 13 may form contact surfaces which provide reliable electrical contact with a measuring device. The insulating coating film 14 may contain two windows parallel to one another that are left blank, each of which bounds arrangements of electrode surfaces. The windows may correspond to the plane-parallel microfluidic measuring chambers 2, 3.

Measuring chambers 2, 3 comprise a first voltammetric three-electrode arrangement 31, a four-electrode conductivity arrangement 33, and a second voltammetric three-electrode arrangement 32. These are described in greater detail below with reference to a preferred embodiment.

In this embodiment, the first measuring chamber 2 contains electrodes 4, 5, 6, 7a,b. Electrodes 4, 5, 6, 7a,b are in each case connected to an analog switch array 55. With the assistance of this analog switch array 55, controlled by a processor 50, electrodes 4, 5, 6, 7a,b can be put to dual use, such that they can be interconnected either to form a first voltammetric three-electrode arrangement 31 with a working electrode AE1 4, a counter-electrode GE1 5 and a reference electrode RE1 6 or to form a four-electrode conductivity arrangement 33 with two current-feeding electrodes 5, 6 and two voltage-tapping electrodes 7a,b. As a result, it is possible to put the first measuring chamber 2 to dual use and thus create a compact measuring zone with a resistance temperature meter 11 on the carrier 1 and in the vicinity of measuring chambers 2, 3.

Electrodes 4, 5, 6, 7a,b are coated with a reagent layer of redox mediator, electrolyte-forming ions and detergents. The redox mediator can make up between 10 and 20 μg of the applied reagent.

The first voltammetric three-electrode arrangement 4, 5, 6 in the first measuring chamber 2 determines the interference concentration of electrochemically active substances in the whole blood sample. The four-electrode conductivity measuring arrangement 5, 6, 7a,b is used in the first measuring chamber 2 to determine a hematocrit value of the whole blood sample via its ionic conductivity.

The second voltammetric three-electrode arrangement 32 in the second measuring chamber 3 further comprises a second voltammetric three-electrode arrangement 32 with a working electrode AE2 8, counter electrode GE2 9 and reference electrode RE2 10 and determines the analyte concentration in the whole blood sample by enzymatic voltammetry. The electrodes can be coated with a reagent system composed of an oxidoreductase and optionally one or more additional catalytically active proteins, electrolyte-forming ions and a redox mediator. The total amount of protein is preferably 50 μg to 80 μg and the electron mediator makes up 40 μg to 80 μg of the applied reagent. Oxidases, peroxidases, or cofactor-dependent dehydrogenases are preferably used as the oxidoreductase. Additional, optionally used, catalytically active proteins are hydrolases, proteases, and esterases. A redox-active metal complex, a quinoid redox dye, or an organometallic compound are preferably used as the redox mediator.

Two feed lines for current feed and two feed lines for voltage tapping are arranged for the serpentine conductor structure 11, also denoted sinuous conductor track, applied in the immediate vicinity of measuring chambers 2, 3, i.e., directly adjacent to measuring chambers 2, 3. The serpentine conductor structure 11 and feed lines 13 may be covered with the insulating coating film 14. The serpentine conductor structure 11 is preferably also not covered with the insulating coating film 14. At 25° C., serpentine resistance may be between 100Ω and 2000Ω.

Not only the feed line ends of the conductivity and resistance-measuring electrodes but also the feed line ends of the two voltammetric three-electrode arrangements are formed as electrical contacts 13 at the opposite end of the carrier 1 of the sensor 100 from the sample-receiving zone 17. As shown schematically in FIG. 2, these enable contact with a measuring device or a processor 50. The processor 50, or the measuring device comprising the processor 50, carries out the inventive method, which is described in greater detail in relation to FIG. 3.

The prestored calibration curves can be saved in a memory/data memory 60 to which the at least one processor 50 is operatively connected.

For example, the measuring device provides the operating voltages required for the respective measuring channels, controls the measurement procedure, processes the measurement signals, displays the measurement result and stores it for example in the memory 60. Such a measuring device can have voltammetric measuring channels, a resistance-measuring channel and a measuring channel for measurement of ionic conductivity. The measuring device can further have an analog switch array 55 for assigning the electrodes according to their function. The polarization voltage for enzymatic amperometric lactate measurement is for example 250 mV and the polarization voltage for amperometric measurement of the electrochemically active substances is +300 mV, in each case relative to the internal reference electrode RE1 and RE2 of the sensor 100. The current feed for four-electrode resistance measurement of the serpentine conductor structure 11 can be operated at 100 μA and the ionic conductivity measurement for determining hematocrit can be carried out with a rectangular, triangular, or sinusoidal AC voltage (f=1000 Hz) with an amplitude 500 mV.

The inventive method is hereinafter described by way of example on the basis of FIG. 3, which method can be performed by a processor 50 integrated on or electrically contacted with the carrier 1.

FIG. 3 shows a schematic representation of a flow chart according to the invention. The determination of a plasma-related analyte value, for example for lactate, glucose etc., which is corrected for the interfering influences hematocrit and electrochemically active substances in the sample and for the influence of ambient temperature, takes place using the sensor 100 according to the invention and the method as described in greater detail in the following embodiment.

In a first step, not explicitly shown here, the at least two measuring chambers 2, 3, see FIG. 1, are filled with a whole blood sample/capillary blood sample via the sample-receiving zone 17 of the sensor. The two measuring chambers 2, 3 are formed on the carrier 1 of the sensor and, as already described above, comprise a first voltammetric three-electrode arrangement 31, a four-electrode conductivity arrangement 33, and a second voltammetric three-electrode arrangement 32.

In one step, after filling of the measuring chambers 2, 3, determination of an ambient temperature S100 is carried out by a temperature-measuring resistor 11 applied to the carrier. Temperature measurement can, for example, take place immediately after filling of the measuring chambers 2, 3 and once time-defined current thresholds of the voltammetric measuring channels have been exceeded. The four-conductor resistance-measuring channel can perform a first temperature-dependent resistance measurement on the serpentine conductor structure 11 over 50 to 1000 ms. 100 to 500 μA direct current can be provided by the measuring channel for current generation by the serpentine conductor structure 11. The measured voltage drop across the serpentine conductor structure 11 at a specified current is used to establish the resistance and can be buffered, for example in memory 60 (see FIG. 2). An ambient temperature can then be established from the resistance.

The ambient temperature can be determined from the linear relationship between the temperature (T) and ohmic resistance (R) of the resistance measurement on the serpentine conductor track 11 according to equation (1) as the average of resistance values measured at the start and end of the measurement procedure. As a result, it is possible to reduce a temperature deviation caused by temperature drift during the measuring process.

$$T = {k}_{temp} * {R}_{serp} + {R}_{serp 0} \qquad ( 1 )$$

in which:
kₜₑₘₚ=the temperature coefficient (temperature sensitivity) of the serpentine conductor track
Rₛₑᵣₚ=the measured serpentine resistance
Rₛₑᵣₚ₀=the serpentine resistance at 0° C.

The temperature coefficient (kₜₑₘₚ) was previously established experimentally using a calibration curve recorded between 0° C. and 50° C. as a function of the resistance value of the serpentine conductor track T=f (Rₛₑᵣₚ), and can be between 0.3 and 0.8° C./Ω. The calibration curve, like all the further prestored calibration curves, can be stored in the memory 60.

In a further step, determination of the ionic conductivity S110 of the whole blood sample is carried out with the four-electrode conductivity arrangement 33.

Determination of the temperature-corrected hematocrit value S210 is further carried out using prestored calibration curves, the measured ambient temperature, and the determined ionic conductivity. Prestored calibration curves and the ambient temperature determined by the temperature-measuring resistor 11 applied to the carrier 1 are used in each case.

For example, in the first measuring chamber 2, counter-electrode GE1 5 and reference electrode RE1 6 for current feed and two voltage-tapping electrodes 7a,b for tapping the voltage drop can be connected via an analog switch array 55 to the four-electrode conductivity measuring channel.

The four-electrode conductivity arrangement 33 can preferably be provided with a sinusoidal AC voltage with a frequency of 100 Hz to 1000 Hz and an amplitude between 100 mV and 1000 mV. The voltage drop resulting as a function of the ionic conductivity of the first measuring chamber is optionally corrected for phase angle and the resistance of the serpentine conductor structure 11 is established. The resistance value can for example be measured and buffered within 0.25 s to 1.0 s after the end of the temperature-dependent resistance measurement.

According to step S210, the hematocrit concentration (Hct) is (provisionally) determined as follows: The resistance or ionic conductivity (G) determined in the first measuring chamber is substantially dependent on the hematocrit, temperature, and optionally concentration of a dissociated analyte or of another endogenous or exogenous metabolite present in dissociated form in the sample. The relationship between the measured resistance (R=1/G) and hematocrit (Hct) is described by a parabolic bundle equation (2):

$$( 2 )$$

$${R}_{Hct} = {f}_{T} ( Hct ) = {k}_{1} ⋆ {conc}_{Hct}^{2} + {k}_{2} * {conc}_{Hct} + {k}_{3} T ∈ \{ 1 ° C . , 5 ° … 45 ° C . \}$$

in which:
R_{Hct}=hematocrit-dependent measured resistance value, established from the ionic conductivity measurement in the first measuring chamber
conc_{Hct}=hematocrit concentration
k₃=resistance of the plasma value (Hct-free blood sample)
k₁ and k₂=cell constants
f_{T} (Hct)=temperature-dependent parabolic bundle

A half-parabolic bundle f_{T} (Hct) required for this purpose is established using blood samples with hematocrit values between for example 0% and 70% for temperatures between preferably 1° C. and 45° C. in, by way of example, at least seven steps. The previously determined ambient temperature is then used to assign the hematocrit-dependent measured resistance value or hematocrit value in the parabolic bundle.

The temperature-corrected hematocrit can be calculated by rearranging equation (2) as follows:

$${conc}_{Hct} = {( {f}_{T} ( Hct ) )}^{- 1} = - {k}_{2} / 2 * {k}_{1} + √ {( - {k}_{2} / 2 * {k}_{1} )}^{2} + ( {R}_{Hct} - {k}_{3} ) / {k}_{1} \qquad ( 3 )$$

The Hct value is linearly interpolated on the basis of the measured temperature for resistance values located between two isothermal Hct half-parabolas of the bundle. A temperature-corrected hematocrit value is thus obtained.

In a further step of the method, voltammetric determination of an interference concentration S120 of electrochemically active substances in the whole blood sample is carried out by the first voltammetric three-electrode arrangement 31. The analog switch array 55, see FIG. 2, can isolate the four electrodes 5, 6, 7a, 7b, 8 from the conductivity measuring channel and interconnect the counter-electrode GE1 5 and reference electrode RE1 6 together with the working electrode 8 as a potentiostatic first voltammetric three-electrode arrangement 31 in the first measuring chamber 2. The electrodes can be connected according to the first voltammetric measuring channel for amperometric measurement of the electrochemically active substances, a polarization voltage between 100 mV and 500 mV being applied to the working electrode.

In a further step, enzymatic-voltammetric determination of an analyte concentration or analyte charge S130 of the whole blood sample is carried out by the second voltammetric three-electrode arrangement 32. In parallel or serially thereto, the second potentiostatic three-electrode arrangement of working electrode AE2 8, counter-electrode GE2 9 and reference electrode RE2 10, which is arranged in the second measuring chamber 3, is set in operation by switching on the second voltammetric measuring channel for enzymatic-voltammetric determination of the analyte concentration. The provided polarization voltage is preferably between 100 mV and 500 mV.

Evaluation can preferably be carried out as follows: The measured signal currents for measurement of analyte concentration and electrochemically active substances is substantially described by the Cottrell equation (4):

$$I ( t ) = z * F * A * √ D / π t * c \qquad ( 4 )$$

in which:
I=current
z=number of electrons transferred
F=Faraday constant (96,485 As/mol)
D=diffusion constant
A=surface area of the working electrode
t=time
c=initial concentration of converted substance

The measurement currents are preferably in each case integrated over time-defined intervals and the resultant charge values are proportional to the analyte concentration or interference concentration (the concentration of interfering substances) and furthermore dependent on temperature and hematocrit.

Determination of a temperature-corrected analyte concentration S230 and the temperature-corrected interference concentration S220 are further carried out using prestored calibration curves, the ambient temperature determined by temperature-measuring resistor applied to the carrier, and the respectively established analyte charge and interference charge. This is described in greater detail below.

Previously recorded curve bundles according to the following functional bundles are used for this purpose:

$${Q}_{TA} = {f}_{T} ( {conc}_{analyte} ) = {k}_{A 1} {conc}_{analyte}^{2} + {k}_{A 2} {conc}_{analyte} + {k}_{A 3} , \qquad ( 5 )$$

$$T ∈ \{ 1 ° C . , 5 ° … 45 ° C . \}$$

$$and$$

$${Q}_{TI} = {f}_{T} ( {conc}_{interf} ) = {k}_{I 1} {conc}_{analyte}^{2} + {k}_{I 2} {conc}_{analyte} + {k}_{I 3} , \qquad ( 6 )$$

$$T ∈ \{ 1 ° C . , 5 ° … 45 ° C . \}$$

and are recorded as a function of the analyte or interferent concentration of the blood samples by way of example for temperatures between 1° C. and 45° C. in at least seven temperature steps at a moderate hematocrit concentration.

In the above equations:
Q_{TA}=temperature-dependent analyte charge values of the curve bundles
Q_{TI}=temperature-dependent interference charge values of the curve bundles
T=ambient temperature (curve bundle parameter)
conc_{analyte}=analyte concentration
conc_{interf}=interferent concentration
k_{A1}, k_{A2}=analyte sensitivity
k_{A3}=charge value at conc_{analyte}=0 mM
k_{I1}, k_{I2}=interferent sensitivity
k_{I3}=charge value at conc_{interf}=0 mM

Functional bundles (5) and (6) can then each be rearranged according to concentration and the concentration values plotted against temperature using a series of closely spaced charge values (n), such that a bundle of n curves is obtained, the functional bundles of which are in each case described by a general quadratic equation (7), (8):

$${conc}_{analyte - Tcorr} = {f}_{QTA} ( T ) = {k}_{AT 1} {T}^{2} + {k}_{AT 2} T + {k}_{AT 3} \qquad ( 7 )$$

$$QTA ∈ \{ 0 , 5 μC , 10 μC … 60 μC \}$$

$${conc}_{interf - Tcorr} = {f}_{QTI} ( T ) = {k}_{IT 1} {T}^{2} + {k}_{IT 2} T + {k}_{IT 3} \qquad ( 8 )$$

$$QTI ∈ \{ 0 , 0.3 μC , 0.6 μC … 6. μC \}$$

in which:
conc_{analyte-Tcorr}=temperature-corrected analyte value
conc_{interf-Tcorr}=temperature-corrected interferent value
QTA=temperature-dependent analyte charge values (curve bundle parameter)
QTI=temperature-dependent interference charge values
(curve bundle parameter)
T=ambient temperature
k_{AT1}, k_{AT2}=temperature sensitivity of analyte measurement
k_{AT3}=analyte concentration value at T=0° C.
k_{IT1}, k_{IT2}=temperature sensitivity of interference measurement
k_{IT3}=interference concentration value at T=0° C.

The point of intersection of the measured charge value and temperature, which is located either on one of the calibration curves of the curve bundle or between two adjacent curves, is used to establish the curve that is closest to this point of intersection.

If the measured charge value for a concentration is not located on a concentration isoline, but between two concentration lines, linear interpolation is carried out.

The functional equation of this curve is used to determine the corresponding concentration of the analytes and the interferents for the standard temperature of preferably 25° C.

In a preferred embodiment, correction of the temperature-corrected hematocrit value to an analyte- and temperature-corrected hematocrit value (S240) can be carried out using prestored calibration curves and the determined temperature-corrected analyte concentration, and/or using prestored calibration curves and the determined temperature-corrected interference concentration.

This is described in greater detail below with reference to an example. The temperature-corrected raw values for analyte and interference concentration are used for correction of the hematocrit value according to equation (9) in order to compensate for the change in conductivity or resistance value due to the presence of an analyte present in dissociated form or of an electrochemically active interfering substance in the sample which can lead to an erroneous hematocrit determination.

The basis for this is a regression line according to equation (7) which was previously recorded as a function of the conductivity or resistance of a dissociated substance concentration in the form of the analyte or of an endogenous or exogenous metabolite:

$${R}_{Hct 25 ° C .} ( = 1 / {G}_{Hct} ) = f ( {C}_{dis} ) = - {a}_{dis} {c}_{dis} + {b}_{dis} \qquad ( 9 )$$

in which:
R_{Hct 25° C.}=hematocrit-dependent measured resistance value established from the ionic conductivity G_{Hct} in the first measurement cell at 25° C.
c_{dis}=concentration of a substance present in dissociated form
−a_{dis}=negative slope of resistance R_{Hct}
b_{dis}=resistance R_{Hct} in the absence of a substance present in dissociated form

The known concentration of a substance to be expected in the blood sample that results in the largest negative slope is used to correct the resistance value to establish the Hct according to equation (10):

$${R}_{Hct - corr} = {a}_{dis} ⋆ {conc}_{RV} + {R}_{Hct 25 ° C .} \qquad ( 10 )$$

in which:
conc_{RV}=raw concentration value of a substance present in dissociated form
a_{dis}=slope of resistance R_{Hct 25° C.} at 25° C. as a function of the concentration of the substance present in dissociated form
R_{Hct-corr}=hematocrit-dependent measured resistance value, corrected for the reduction in resistance value due to the presence of a substance present in dissociated form at 25° C.

In a further step, correction of the temperature-corrected analyte concentration and the temperature-corrected interference concentration to a plasma-related hematocrit- and temperature-corrected analyte concentration (S330) and a plasma-related hematocrit- and temperature-corrected interference concentration (S320) is carried out, in each case using prestored calibration curves and the previously determined temperature-corrected hematocrit value or the analyte- and temperature-corrected hematocrit value.

The established hematocrit value, which is itself corrected for temperature and optionally for the presence of ionically conductive substances, is used below to correct the analyte concentration value and the concentration value of electrochemically active substances (interferent concentration). In other embodiments, only the temperature-corrected hematocrit value is used, if for example no significant additional contribution from ionically conductive substances is to be expected. This is achieved using a previously recorded line bundle according to equations (11) and (12), which is recorded as a function of hematocrit, for example for a range from 0% to 70% or the corresponding Hct-dependent resistance value, in at least seven steps at a temperature of 25° C. against a reference system:

$${Q}_{Hct analyte} = {f}_{Hct} ( {conc}_{analyte} ) = {a}_{A} {conc}_{analyte temp corr} + {b}_{A} \qquad ( 11 )$$

$$Hct ∈ \{ 0 \% , 20 … 0 \% \}$$

$$and$$

$${Q}_{Hct interf} = {f}_{Hct} ( {conc}_{interf} ) = {a}_{interf} {conc}_{interf temp corr} + {b}_{interf} \qquad ( 12 )$$

$$Hct ∈ \{ 0 \% , 20 \% … 70 \% \}$$

in which:
Hct=hematocrit value (line bundle parameter of the temperature-corrected analyte concentration curves and interference substance concentration curves)
Q_{Hct analyte}=hematocrit-dependent charge values of the temperature-corrected analyte concentration values
Q_{Hct interf}=hematocrit-dependent charge values of the temperature-corrected analyte concentration values of the interferent concentration values
conc_{analyte} temp corr=temperature-corrected analyte concentration values
conc_{interf} temp corr=temperature-corrected interference concentration values
a_{A}=slope (analyte sensitivity)
b_{A}=constant, charge value at c_{analyte}=0 mM
a_{interf}=slope (interferent sensitivity)
b_{interf}=constant, charge value at c_{interf}=0 mM

On the basis of functional equations (11), (12), the respective concentration values can be established from the measured charge values Q_{Hct-analyte} and Q_{Hct-interf} and plotted against hematocrit concentration using a series of closely spaced charge values (n), such that a bundle of n curves is obtained, each of which is described by a general quadratic equation (13), (14):

$${conc}_{analyte - T + Hct corr} = {f}_{QTHct - A} ( Hct ) = {k}_{THct A 1} {Hct}^{2} + {k}_{THct A 2} Hct + {k}_{THct A 3} {Q}_{THct - A} ∈ \{ 0 μ C , 5 μ C , 10 μ C … 60 μ C \} \qquad ( 13 )$$

$${conc}_{interf - T + Hct corr} = {f}_{QTHct - I} ( Hct ) = {k}_{THct I 1} {Hct}^{2} + {k}_{THct I 2} Hct + {k}_{THct I 3} {Q}_{THct - I} ∈ \{ 0 μ C , 0.3 μ C , 0.6 μ C … 6. μ C \} \qquad ( 14 )$$

in which:
conc_{analyte-T+Hct corr}=temperature- and hematocrit-corrected analyte value
conc_{interf-T+Hct corr}=temperature- and hematocrit-corrected interferent value
QTHct-A=temperature-compensated hematocrit-dependent analyte charge (curve bundle parameter)
QTHct-I=temperature-compensated hematocrit-dependent interference charge (curve bundle parameter)
Hct=temperature-corrected hematocrit value
k_{THctl A1}, k_{THct A2}=temperature-compensated hematocrit sensitivity of the analyte measurement
k_{THctl I1}, k_{THct I2}=temperature-compensated hematocrit sensitivity of the interference measurement
k_{THct A3}, k_{THct I3}=concentration values at Hct=0% (plasma value)

The point of intersection of the measured charge value and hematocrit, which is located either on one of the curves of the curve bundle or between two adjacent curves, is used to establish the curve that is closest to this point of intersection.

If the measured charge value for a concentration is not located on a charge isoline, but between two charge lines, linear interpolation is carried out.

The functional equation of this curve is used to calculate the corresponding concentration of the analytes and the interferents for a hematocrit concentration of 0% (plasma value).

In a further step of the method, determination of the analyte concentration (S430) is carried out by subtracting the hematocrit- and temperature-corrected interference concentration from the hematocrit- and temperature-corrected analyte concentration.

This is described in greater detail below. In this evaluation step, the influence of electrochemically active substances on the determination of the analyte concentration value can be corrected by subtraction and taking account of a sensitivity factor according to equation (15):

$${conc}_{analyte \_ T + Hct + interf - corr} = {conc}_{analyte \_ T + Hct - corr} - {C}_{int \_ T + Hct corr .} * {K}_{s} \qquad ( 15 )$$

in which:
conc_{analyte_T+Hct+interf-corr}=plasma-related analyte concentration, corrected for the influence of ambient temperature, hematocrit and electrochemically active substances
conc_{analyte_T+Hct-corr}=plasma-related analyte concentration, corrected for the influence of ambient temperature and hematocrit
C_{int_T+Hct corr.}=Plasma-related combined concentration of electrochemically active substances, corrected for the influence of ambient temperature and hematocrit
Kₛ=sensitivity factor (quotient of sensitivity of the amperometric analyte measuring system and of the amperometric interference measuring system to electrochemically active substances)

In specific embodiments, Kₛ=1 can also be set if a similar reagent composition is assumed in both measuring chambers.

In this way, the sensor and method according to the invention enable the correction of mutually influencing parameters and thus also a more accurate procedure for correction of the analyte value. The basis for this is also the very accurate determination ambient temperature directly on the carrier in the vicinity of the measuring chambers.

### Exemplary Embodiment

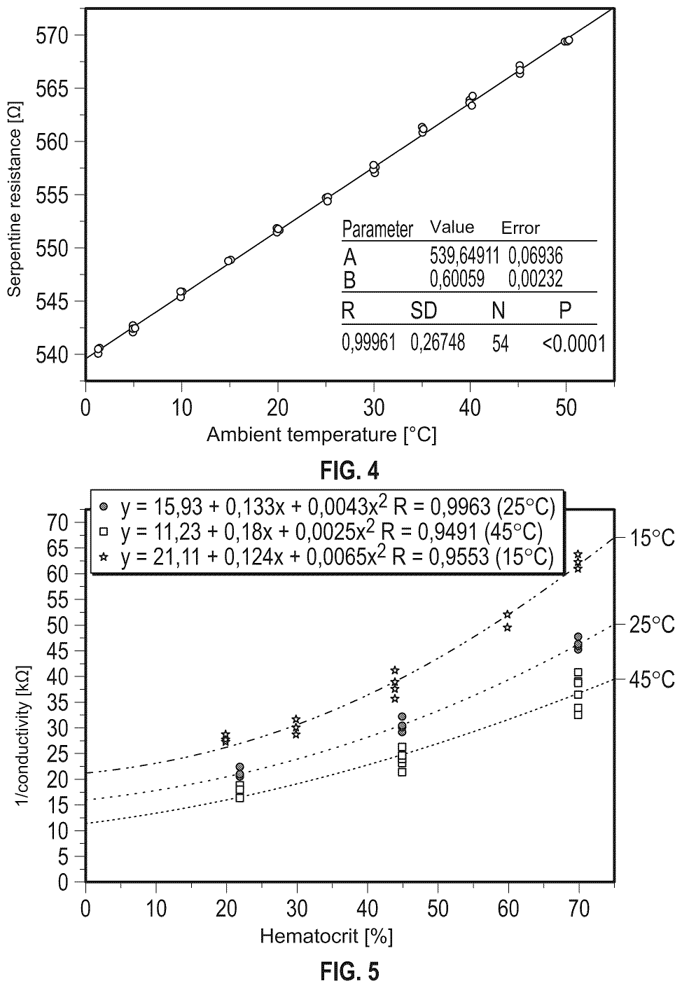

The following FIGS. 4 to 10 demonstrate the above-described method and the sensor that performs the method on the basis of a specific example with lactate as the analyte to be determined in whole blood, reference being made for supplementary steps to the procedure described above in relation to FIG. 3. The specific exemplary embodiment also supplements the above statements.

For determining lactate, electrodes 4, 5, 6 of the first measuring chamber 2 within the first measuring window can preferably be coated with a reagent layer of redox mediator (20 μg/mL), sodium chloride (2 mM), Tergitol (0.3% V/V), and CMC (0.5% W/V). This enables both a quantitative combined measurement of electrochemically active substances (interferents) in the sample using the first voltammetric three-electrode arrangement 31 and the hematocrit-dependent ionic conductivity measurement using the four-electrode conductivity arrangement 33.

Electrodes 8, 9, 10 within the second measuring chamber 3 are preferably coated for lactate with a reagent solution of a lactate oxidase (2 μg/mL), sodium chloride (50 mM), CMC (0.5% W/V), Tergitol (0.3% V/V), and ferricyanide (100 μg/mL) as redox mediator. The total volume of the applied reagent is preferably 200 nL. The serpentine resistance of the temperature-measuring resistor 11 is, purely by way of example, 560 Ohm at 25° C. and has a temperature dependence of 0.6Ω/° C.; see FIG. 4.

The feed line ends of both the conductivity and the resistance-measuring electrodes as well as the feed line ends of the two voltammetric three-electrode arrangements are formed as contact surfaces 13 at the opposite end of the carrier 1 from the sample-receiving zone 17. These contact surfaces 13 enable contact with a processor 50, see FIG. 2. The processor 50 may be part of a connected measuring device, as described in connection with FIGS. 1 and 2.

Immediately after filling of the measuring chambers with a capillary blood sample and once time-defined current thresholds of the voltammetric measuring channels have been exceeded, a first temperature-dependent resistance measurement, preferably over 500 ms, is carried out on the four-conductor arrangement of the serpentine conductor structure 11. In the serpentine conductor structure 11, a 100 μA direct current is fed in via the measuring channel as the excitation signal. The measured resistance value, in this specific case 549.1Ω, is buffered. In other embodiments, this resistance value can be used directly. This step corresponds to step S100 in FIG. 3.

After the resistance measurement, counter-electrode GE 5 and reference electrode RE1 6 provided for current feed and the two voltage-tapping electrodes 7a,b of the first measuring chamber 2 are connected to the four-electrode conductivity measuring channel of the measuring device via analog switches of the device's internal analog switch array 55, see FIG. 2. Working electrode AE1 4 is switched off. The conductivity measuring channel can, for example, provide a rectangular AC voltage with a frequency of 1000 Hz and an amplitude of 100 mV. The resistance value measured and averaged over a period of one second is 40 kΩ and is buffered. As a result, the ionic conductivity is determined, see S110.

Using the measuring device's analog switches, the voltage-tapping electrodes are then isolated from the measuring channel, and counter-electrode GE1 5 and reference electrode RE1 6 used for current feed together with working electrode AE1 4 are interconnected as a potentiostatic three-electrode arrangement in the first measuring chamber 2 and connected to the first voltammetric measuring channel for amperometric measurement of the electrochemically active substances, a polarization voltage of 300 mV being applied to the working electrode AE1 4. In parallel, the second potentiostatic three-electrode arrangement of working electrode AE2 8, GE 2 9 and RE2 10 for enzymatic amperometric determination of the analyte concentration in the second measuring chamber is set in operation by switching on the second voltammetric measuring channel that applies a polarization voltage of 200 mV to working electrode AE2 8.

In this example, for example, three seconds after the polarization voltages are switched on, the signal currents of both measuring channels are measured over a period of five seconds, integrated and the raw charge values Q_{conc}=12.8 μC and Q_{interf} 1.2 μC are buffered. These charges contain information about the analyte concentration and the interferent concentration. This step corresponds to steps S130 and S120. The electrodes in the two measuring chambers are then isolated from the measuring channels via the analog switch array.

A second resistance measurement is then carried out on the serpentine conductor track over a further 500 ms, a resistance value of 548.4Ω being obtained. The resistance values measured at the beginning and end, 549.1Ω and 548.4Ω, are averaged, preferably arithmetically. The averaged resistance value of 548.76Ω corresponds to a temperature of 15.2° C. according to equation (1) in accordance with the calibration curve in FIG. 4. This temperature value is buffered and used for the further evaluation steps.

The calibration curves in FIG. 5 illustrate the dependence between ionic conductivity or resistance and hematocrit by way of example for three different temperatures. The resistance value of 40 kΩ, averaged as a function of the ionic conductivity of the first measuring chamber 2 and over an exemplary period of one second, is established using equation 3 and corresponds to a hematocrit of 45% at 15° C., see step S210 in FIG. 3. The correlation curve or calibration curve between resistance value/ionic conductivity and hematocrit at 15° C. which is obtained from the empirically recorded and prestored values for 15° C. in FIG. 5, is defined according to equation (16)

$$Y = 21.11 + 0.1 \; 24 x + 0.0 \; 065 {x}^{2} \qquad ( 16 )$$

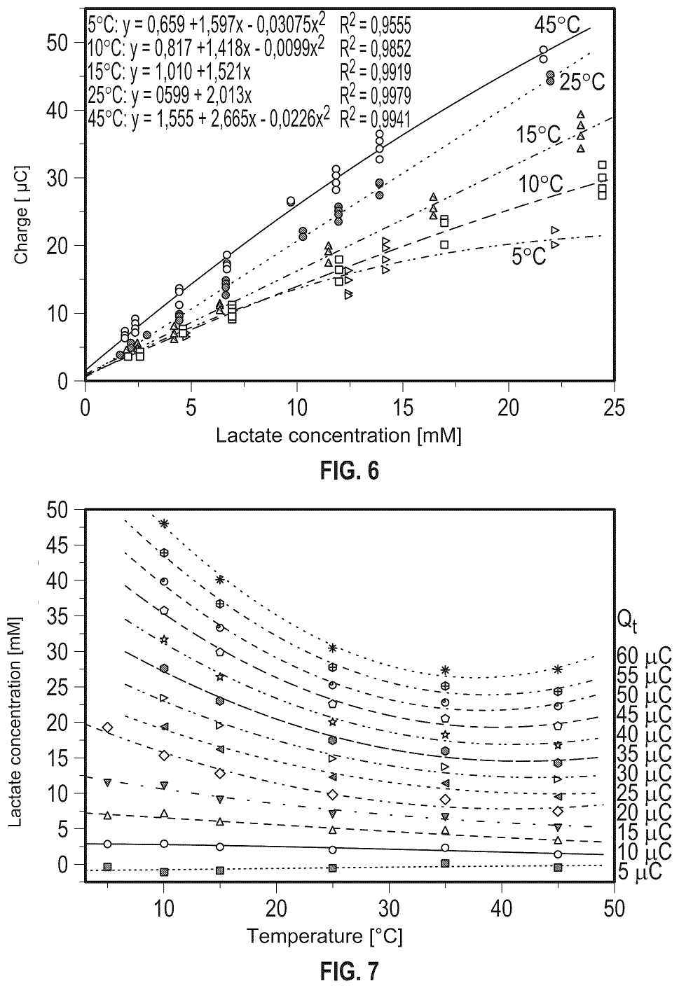

In a subsequent step, the signal currents for measurement of the analyte concentration and electrochemically active substances have to be corrected for the influence of temperature, see steps S220 and S230 in FIG. 3. For the charge value of 12.8 μC for lactate, which is buffered as the charge after integration, the point of intersection with the nominal temperature of 25 C is established using the curve bundle in FIG. 7, which was generated from the bundle of calibration curves in FIG. 6, this point of intersection being located between the 10 μC and 15 μC charge value curves and corresponding to a lactate value of approx. 6.5 mM on graphical determination. The two curves adjacent to the established charge value of 12.8 PC, each with the same charges of 10 μC and 15 μC, are defined according to equations (17) and (18):

$${Q}_{10 μ C} : y = 7.52 \; 768 - 0.10833 x + 0.0 \; 0035 {x}^{2} \qquad ( 17 )$$

$${Q}_{15 μ C} : y = 13.10 \; 747 - 0.27714 x + 0.0 \; 0237 {x}^{2} \qquad ( 18 )$$

At a nominal temperature of 25° C., equations (17), (18) give Q_{10 μC}=5.04 mM and Q_{15 μC}=7.69 mM. After linear iteration, the charge value of 12.8 μC corresponds at a nominal temperature of 25° C. to a lactate concentration of 6.52 mM. A concentration value corrected to 25° C. of 0.6 mM for electrochemically active substances is determined in a similar manner and as described in greater detail in connection with FIG. 3.

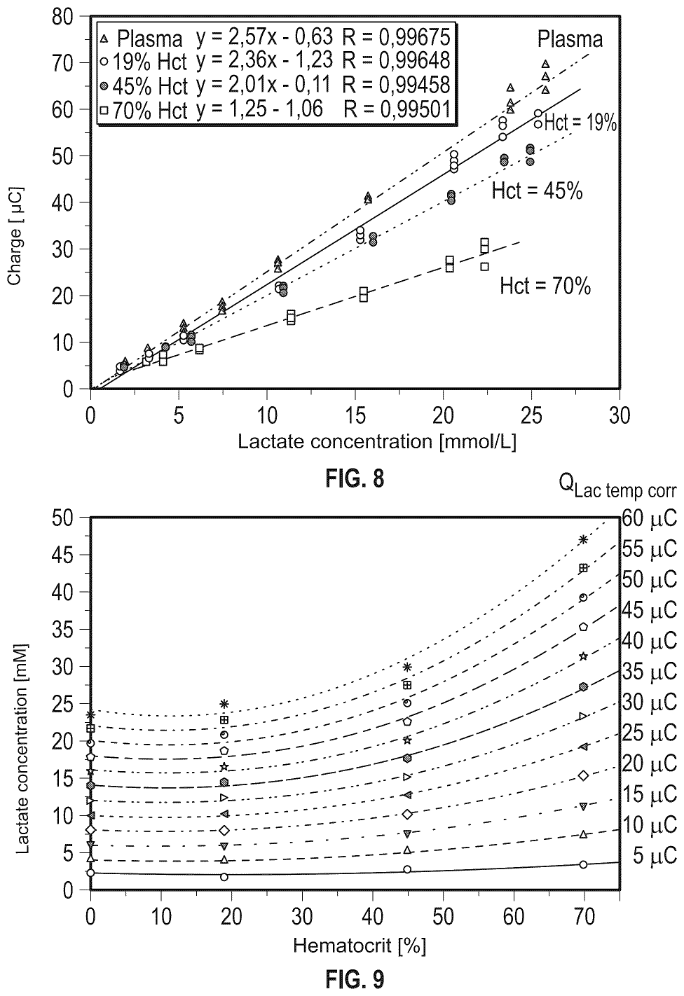

The temperature-corrected raw concentration values are now the basis, using the previously recorded and prestored calibration curves as shown in FIG. 8 and FIG. 9 and according to equations (11) to (14), for correction of the hematocrit dependence of the lactate concentration value, see S330 in FIG. 3.

The point of intersection with the previously determined hematocrit value of 45% is established for the buffered, temperature-corrected lactate concentration value of 6.52 mM using the prestored curve bundle in FIG. 9, which was generated from the bundle of calibration curves (charge values vs. lactate concentration) as a function of hematocrit (FIG. 8). In the present example, this point is located between the curves with the same charges of 10 μC and 15 μC. The two curves Q₁₀ and Q₁₅ from FIG. 9 are described by equations (19) and (20):

$${Q}_{10 μ C} : Y = 4.0649 - 0.02965 x + 0.0 \; 0106 {x}^{2} \qquad ( 19 )$$

$${Q}_{15 μ C} : Y = 6.07069 - 0.04224 x + 0.0 \; 0164 {x}^{2} \qquad ( 20 )$$

If x=0% is used for hematocrit, in order to obtain the plasma-related value, plasma lactate values of 4.06 mM and 6.07 mM are obtained. Using the lactate values for 45%, a linear iteration is carried out, such that a hematocrit- and temperature-corrected plasma lactate value of 5.4 mM was obtained in this example.

A similar procedure was used for the interference concentration value of electrochemically active substances corrected to 25° C. and, following hematocrit correction, a concentration of 0.4 mM was obtained for the hematocrit- and temperature-corrected interference concentration. These steps correspond to steps S330 and S320 described above in FIG. 3.

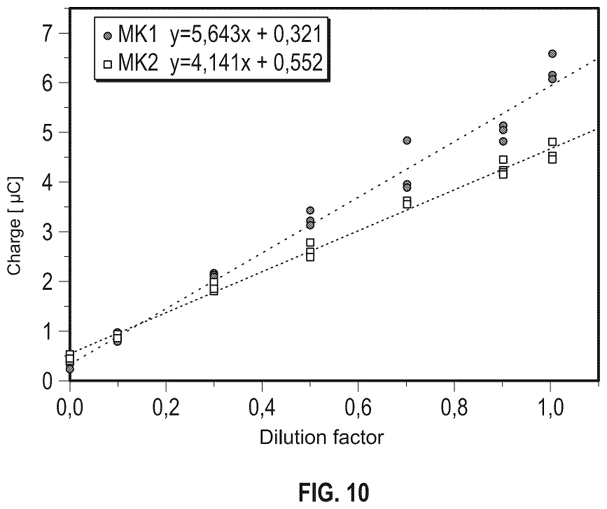

In a further step, the influence of the electrochemically active substances on the determination the analyte concentration value is corrected by subtraction according to step S430 in FIG. 3. A weighting/sensitivity factor Kₛ according to equation (15) can also be taken into account, this factor being a quotient of the sensitivity of the amperometric analyte measuring system and of the amperometric interference measuring system to electrochemically active substances. Kₛ is calculated from the quotient of the gradient of the linear equation for MK2 and the gradient of the linear equation for MK1 from FIG. 10 and in each case reflects the influence of electrochemically active substances on the charge values from measuring chambers 1 and 2 respectively. The differing sensitivity of the measuring chambers to electrochemically active substances is caused by the different reagent composition. Since the reagent mixture for lactate measurement in the second measuring chamber 3 generally has a higher ion concentration and protein content than the reagent system for sensing electrochemically active substances in the first measuring chamber, electrochemically active substances are detected with greater sensitivity in the first measuring chamber. In the present case, a sensitivity factor of Kₛ=0.74 was determined using the calibration curves in FIG. 10, such that in this example the plasma-related lactate value corrected for the influence of electrochemically active interfering substances is 5.1 mM.

The described method is in particular suitable for single-use sensors that are intended to provide clinically relevant and accurately plasma-related concentration values. Thanks to the use of a temperature-measuring resistor applied to, i.e., integrated on, the carrier, it is possible to measure the current ambient temperature without interference in comparison with the prior art, see the explanations above. This accurately measured ambient temperature is in turn used in stages to systematically correct for interference from hematocrit and electrochemically active substances. As a result, interfering influences can be systematically eliminated, such that they can be adequately corrected. Because the analyte concentration measurement is plasma-related, the sensor is particularly suitable for emergency applications or for rapidly required analyte determinations in which whole blood has to be used.

### LIST OF REFERENCE SIGNS

Reference signs used herein include the following:
100 Sensor
1 Carrier
2 First measuring chamber
3 Second measuring chamber
4 Working electrode AE1
5 Counter-electrode
6 Reference electrode
7a, 7b Voltage-tapping electrodes
8 Working electrode
9 Counter-electrode
10 Reference electrode
11 Temperature-measuring resistor/serpentine conductor structure
12 Feed lines
13 Electrical contacts
14 Insulating coating
17 Sample-receiving zone
18a,b Ventilation line
D Carrier portion
L Total length
31 First voltammetric three-electrode arrangement
32 Second voltammetric three-electrode arrangement
33 Four-electrode conductivity arrangement
50 Processor unit
55 Analog switch array
60 Memory/data memory

### EXAMPLES

Examples of embodiments described above include the following:

1. A method for determining a plasma-related analyte concentration in whole blood by a sensor comprising the steps of:
filling of at least two measuring chambers with a whole blood sample via a sample-receiving zone of the sensor, wherein at least two measuring chambers are formed on a carrier of the sensor and comprise a first voltammetric three-electrode arrangement, a four-electrode conductivity arrangement, and a second voltammetric three-electrode arrangement;
after filling of the measuring chambers, determination of an ambient temperature by a temperature-measuring resistor applied to the carrier;
determination of the ionic conductivity of the whole blood sample with the four-electrode conductivity arrangement;
voltammetric determination of an interference charge of electrochemically active substances in the whole blood sample by the first voltammetric three-electrode arrangement;
enzymatic-voltammetric determination of an analyte charge in the whole blood sample by the second voltammetric three-electrode arrangement;
determination of a temperature-corrected analyte concentration using prestored calibration curves, the determined ambient temperature, and the determined analyte charge;
determination of a temperature-corrected interference concentration using the prestored calibration curves, the determined ambient temperature and the determined interference charge; and determination of a temperature-corrected hematocrit value using prestored calibration curves, the determined ambient temperature, and the determined ionic conductivity;
correction of the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value;
determination of the analyte concentration by subtracting the hematocrit- and temperature-corrected interference concentration from the hematocrit- and temperature-corrected analyte concentration.

2. The method according to Example 1, further comprising correction of the temperature-corrected hematocrit value to an analyte- and temperature-corrected hematocrit value using prestored calibration curves and the determined temperature-corrected analyte concentration, and/or using prestored calibration curves and the determined temperature-corrected interference concentration.

3. The method according to any one of Examples 1 to 2, wherein the temperature-measuring resistor is a serpentine conductor structure that is applied to the carrier of the sensor.

4. The method according to Example 3, wherein the serpentine conductor structure is positioned adjacent to the measuring chambers.

5. The method according to any one of Examples 3 to 4, wherein the serpentine conductor structure is positioned in a carrier portion that is less than one third, preferably less than one quarter, and still more preferably less than one fifth of the total length of the carrier.

6. The method according to any one of Examples 1 to 5, wherein determination of the ambient temperature comprises the following steps:
determination of a first temperature after filling of the measuring chambers;
determination of a second temperature after determination of the ionic conductivity, the analyte charge, and/or of the interference charge of electrochemically active substances in the whole blood sample; and
determination of the ambient temperature by arithmetic averaging from the determined temperatures.

7. The method according to any one of Examples 1 to 6, wherein the first measuring chamber comprises the first voltammetric three-electrode arrangement and the four-electrode conductivity arrangement, and the second measuring chamber comprises a second voltammetric three-electrode arrangement, further comprising switching of the electrodes in the first measuring chamber between the three-electrode arrangement for voltammetric measurement and the four-electrode conductivity arrangement for measurement of ionic conductivity using an integrated or reversibly connected analog switch array.

8. The method according to any one of Examples 1 to 7, wherein the first three-electrode and four-electrode conductivity arrangements have a reagent coating comprising a redox mediator, and wherein the second three-electrode arrangement has a reagent coating comprising an oxidoreductase or further catalytically active proteins, and a redox mediator.

9. The method according to any one of Examples 1 to 8, wherein determination of the ionic conductivity of the whole blood sample is carried out by the four-electrode conductivity arrangement, voltammetric determination of the interference charge of electrochemically active substances in the whole blood sample by the first voltammetric three-electrode arrangement, and enzymatic-voltammetric determination of the analyte concentration of the whole blood sample by the second voltammetric three-electrode arrangement in steps within a measuring interval of 8 s to 20 s and preferably between 8 and 11 s.

10. A sensor for determination of plasma-related analyte concentration in whole blood comprising:
at least two measuring chambers that are fillable with a whole blood sample via a sample-receiving zone of the sensor, wherein the at least two measuring chambers are formed on a carrier and comprise a first voltammetric three-electrode arrangement, a four-electrode conductivity arrangement, and a second voltammetric three-electrode arrangement,
a temperature-measuring resistor applied to the carrier for determination of an ambient temperature;
at least one processor unit that is reversibly connected to the sensor via an electrical contacting means or is integrated on the carrier, and is set up to do the following:
   control the temperature-measuring resistor in order to determine the ambient temperature after filling of the measuring chambers;
   control the four-electrode conductivity arrangement in order to determine an ionic conductivity of the whole blood sample;
   control the first three-electrode arrangement in order to voltammetrically determine an interference charge of electrochemically active substances in the whole blood sample;
   control the second three-electrode arrangement in order to enzymatic-voltammetrically determine an analyte charge of the whole blood sample;
   determine a temperature-corrected analyte concentration using prestored calibration curves, the determined ambient temperature, and the determined analyte charge; determine a temperature-corrected interference concentration using prestored calibration curves, the determined ambient temperature, and the determined interference charge; and determine a temperature-corrected hematocrit value using prestored calibration curves, the determined ambient temperature, and the determined ionic conductivity;
   correct the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value; and
determine the analyte concentration by subtracting the hematocrit- and temperature-corrected interference concentration from the hematocrit- and temperature-corrected analyte concentration.

11. The sensor according to Example 10, wherein the temperature-measuring resistor is a serpentine conductor structure that is applied to the carrier of the sensor.

12. The sensor according to Example 11, wherein the serpentine conductor structure is positioned adjacent to the measuring chambers.

13. The sensor according to any one of Examples 11 to 12, wherein the serpentine conductor structure positioned in a carrier portion that is less than one third, preferably less than one quarter, and still more preferably less than one fifth of the total length of the carrier.

14. The sensor according to any one of Examples 10 to 13, wherein the first measuring chamber comprises the first voltammetric three-electrode arrangement and the four-electrode conductivity arrangement, and the second measuring chamber comprises the second voltammetric three-electrode arrangement, wherein the at least one processor unit is set up to switch electrodes in the first measuring chamber between the first three-electrode arrangement for voltammetric measurement and the four-electrode conductivity arrangement for measurement of ionic conductivity using an integrated or reversibly connected analog switch array.

What is claimed is:

1. A method for determining a plasma-related analyte concentration in whole blood by a sensor comprising:
filling of at least two measuring chambers with a whole blood sample via a sample-receiving zone of the sensor, wherein at least two measuring chambers are formed on a carrier of the sensor and comprise a first voltammetric three-electrode arrangement, a four-electrode conductivity arrangement, and a second voltammetric three-electrode arrangement;
after filling of the measuring chambers, determination of an ambient temperature by a temperature-measuring resistor applied to the carrier;
determination of ionic conductivity of the whole blood sample with the four-electrode conductivity arrangement;
voltammetric determination of an interference charge of electrochemically active substances in the whole blood sample by the first voltammetric three-electrode arrangement;
enzymatic-voltammetric determination of an analyte charge in the whole blood sample by the second voltammetric three-electrode arrangement;
determination of a temperature-corrected analyte concentration using prestored calibration curves, the determined ambient temperature, and the determined analyte charge;
determination of a temperature-corrected interference concentration using the prestored calibration curves, the determined ambient temperature and the determined interference charge; and determination of a temperature-corrected hematocrit value using prestored calibration curves, the determined ambient temperature, and the determined ionic conductivity;
correction of the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value;
determination of the analyte concentration by subtracting the hematocrit- and temperature-corrected interference concentration from the hematocrit- and temperature-corrected analyte concentration.

2. The method according to claim 1, wherein the temperature-measuring resistor is a serpentine conductor structure that is applied to the carrier of the sensor.

3. The method according to claim 1, further comprising correction of the temperature-corrected hematocrit value to an analyte- and temperature-corrected hematocrit value using prestored calibration curves and the determined temperature-corrected analyte concentration, and/or using prestored calibration curves and the determined temperature-corrected interference concentration.

4. The method according to claim 3, wherein the temperature-measuring resistor is a serpentine conductor structure that is applied to the carrier of the sensor.

5. The method according to claim 4, wherein the serpentine conductor structure is positioned in a carrier portion that is less than one third of the total length of the carrier.

6. The method according to claim 4, wherein the serpentine conductor structure is positioned adjacent to the measuring chambers.

7. The method according to claim 6, wherein the serpentine conductor structure is positioned in a carrier portion that is less than one third of the total length of the carrier.

8. The method according to claim 1, wherein determination of the ambient temperature comprises:
determination of a first temperature after filling of the measuring chambers;
determination of a second temperature after determination of the ionic conductivity, the analyte charge, and/or of the interference charge of electrochemically active substances in the whole blood sample; and
determination of the ambient temperature by arithmetic averaging from the determined temperatures.

9. The method according to claim 1, wherein the first measuring chamber comprises the first voltammetric three-electrode arrangement and the four-electrode conductivity arrangement, and the second measuring chamber comprises a second voltammetric three-electrode arrangement, further comprising switching of electrodes in the first measuring chamber between the three-electrode arrangement for voltammetric measurement and the four-electrode conductivity arrangement for measurement of ionic conductivity using an integrated or reversibly connected analog switch array.

10. The method according to claim 1, wherein the first three-electrode and four-electrode conductivity arrangements have a reagent coating comprising a redox mediator, and wherein the second three-electrode arrangement has a reagent coating comprising an oxidoreductase or further catalytically active proteins, and a redox mediator.

11. The method according to claim 1, wherein determination of the ionic conductivity of the whole blood sample is carried out by the four-electrode conductivity arrangement, voltammetric determination of the interference charge of electrochemically active substances in the whole blood sample by the first voltammetric three-electrode arrangement, and enzymatic-voltammetric determination of the analyte concentration of the whole blood sample by the second voltammetric three-electrode arrangement in steps within a measuring interval of 8 s to 20 s.

12. A sensor for determination of plasma-related analyte concentration in whole blood comprising:
at least two measuring chambers that are fillable with a whole blood sample via a sample-receiving zone of the sensor, wherein the at least two measuring chambers are formed on a carrier and comprise a first voltammetric three-electrode arrangement, a four-electrode conductivity arrangement, and a second voltammetric three-electrode arrangement,
a temperature-measuring resistor applied to the carrier for determination of an ambient temperature;
at least one processor unit that is reversibly connected to the sensor via an electrical contacting means or is integrated on the carrier, and is set up to:
control the temperature-measuring resistor in order to determine the ambient temperature after filling of the measuring chambers;
control the four-electrode conductivity arrangement in order to determine an ionic conductivity of the whole blood sample;
control the first three-electrode arrangement in order to voltammetrically determine an interference charge of electrochemically active substances in the whole blood sample;
control the second three-electrode arrangement in order to enzymatic-voltammetrically determine an analyte charge of the whole blood sample;
determine a temperature-corrected analyte concentration using prestored calibration curves, the determined ambient temperature, and the determined analyte charge; determine a temperature-corrected interference concentration using prestored calibration curves, the determined ambient temperature, and the determined interference charge; and determine a temperature-corrected hematocrit value using prestored calibration curves, the determined ambient temperature, and the determined ionic conductivity;
correct the temperature-corrected analyte concentration and the temperature-corrected interference concentration to a plasma-related hematocrit- and temperature-corrected analyte concentration and a plasma-related hematocrit- and temperature-corrected interference concentration, in each case using prestored calibration curves and the previously determined temperature-corrected hematocrit value; and
determine the analyte concentration by subtracting the hematocrit- and temperature-corrected interference concentration from the hematocrit- and temperature-corrected analyte concentration.

13. The sensor according to claim 12, wherein the temperature-measuring resistor is a serpentine conductor structure that is applied to the carrier of the sensor.

14. The sensor according to claim 13, wherein the first measuring chamber comprises the first voltammetric three-electrode arrangement and the four-electrode conductivity arrangement, and the second measuring chamber comprises the second voltammetric three-electrode arrangement, wherein the at least one processor unit is set up to switch electrodes in the first measuring chamber between the first three-electrode arrangement for voltammetric measurement and the four-electrode conductivity arrangement for measurement of ionic conductivity using an integrated or reversibly connected analog switch array.

15. The sensor according to claim 13, wherein the serpentine conductor structure is positioned in a carrier portion that is less than one fifth of the total length of the carrier.

16. The sensor according to claim 15, wherein the first measuring chamber comprises the first voltammetric three-electrode arrangement and the four-electrode conductivity arrangement, and the second measuring chamber comprises the second voltammetric three-electrode arrangement, wherein the at least one processor unit is set up to switch electrodes in the first measuring chamber between the first three-electrode arrangement for voltammetric measurement and the four-electrode conductivity arrangement for measurement of ionic conductivity using an integrated or reversibly connected analog switch array.

17. The sensor according to claim 13, wherein the serpentine conductor structure is positioned adjacent to the measuring chambers.

18. The sensor according to claim 17, wherein the first measuring chamber comprises the first voltammetric three-electrode arrangement and the four-electrode conductivity arrangement, and the second measuring chamber comprises the second voltammetric three-electrode arrangement, wherein the at least one processor unit is set up to switch electrodes in the first measuring chamber between the first three-electrode arrangement for voltammetric measurement and the four-electrode conductivity arrangement for measurement of ionic conductivity using an integrated or reversibly connected analog switch array.

19. The sensor according to claim 17, wherein the serpentine conductor structure is positioned in a carrier portion that is less than one fifth of the total length of the carrier.

20. The sensor according to claim 19, wherein the first measuring chamber comprises the first voltammetric three-electrode arrangement and the four-electrode conductivity arrangement, and the second measuring chamber comprises the second voltammetric three-electrode arrangement, wherein the at least one processor unit is set up to switch electrodes in the first measuring chamber between the first three-electrode arrangement for voltammetric measurement and the four-electrode conductivity arrangement for measurement of ionic conductivity using an integrated or reversibly connected analog switch array.
